(19) United States
(12) Patent Application Publication
(10) Pub. No.: US 2009/0136436 A1
(43) Pub. Date: May 28, 2009

(54) FRAGRANCING COMPOSITION COMPRISING THE COMBINATION OF A HYDROXYAMINOBENZOPHENONE SCREEN A, A CINNAMATE SCREEN B AND A PIPERIDINOL, BENZOTRIAZOLE OR DIBENZOYLMETHANE COMPOUND C

(75) Inventor: ISABELLE BARA, La Varenne St. Hilaire (FR)

Correspondence Address: BUCHANAN, INGERSOLL & ROONEY PC, POST OFFICE BOX 1404, ALEXANDRIA, VA, 22313-1404, US

(73) Assignee: L'OREAL, Paris (FR)

(21) Appl. No.: 12/124,865

(22) Filed: May 21, 2008

Related U.S. Application Data
(60) Provisional application No. 60/929,002, filed on Jun. 7, 2007.
(60) Provisional application No. 60/929,003, filed on Jun. 7, 2007.
(60) Provisional application No. 60/929,004, filed on Jun. 7, 2007.
(60) Provisional application No. 60/929,005, filed on Jun. 7, 2007.

(30) Foreign Application Priority Data
May 21, 2007 (FR) 0755152
May 21, 2007 (FR) 0755153
May 21, 2007 (FR) 0755154
May 21, 2007 (FR) 0755155

(51) Int. Cl. A61K 8/49; A61Q 17/04

(52) U.S. Cl. 424/59

(57) ABSTRACT

The invention relates to a fragrancing composition comprising, in a cosmetically acceptable medium:
a) at least 2% by weight of a fragrancing substance, relative to the total weight of the composition;
b) at least one hydroxyaminobenzophenone screen A of specific formula (I) that will be defined below in detail;
c) at least one cinnamate screen B;
d) at least one compound C selected from among:
   (i) a piperidinol compound of specific formula (II) that will be defined below in detail;
   (ii) at least one benzotriazole compound of specific formula (III) that will be defined below in detail;
   (iii) a dibenzoylmethane UV screen, optionally in combination with at least one salicylate screen D.

The invention also relates to the use of this combination b), c) and d) in a fragrancing composition comprising, in a cosmetically acceptable medium, at least 2% by weight of a fragrancing substance, relative to the total weight of the composition, as an agent for stabilizing the organoleptic properties of the composition with respect to outside attacks such as the light or temperature differences, in particular the color and/or the odor of said composition.

The invention also relates to a cosmetic process for fragrancing human keratin materials, comprising the application of said fragrancing composition to the keratin materials.

### CROSS-REFERENCE TO PRIORITY/PROVISIONAL APPLICATIONS

This application claims priority under 35 U.S.C. §119 of FR 07/55152, May 21, 2007; FR 07/55153, filed May 21, 2007; FR 07/55154, filed May 21, 2007; and FR 07/55155, filed May 21, 2007; and of U.S. Provisional Application No. 60/929,002, filed Jun. 7, 2007, U.S. Provisional Application No. 60/929,003, filed Jun. 7, 2007, U.S. Provisional Application No. 60/929,004, filed Jun. 7, 2007 and U.S. Provisional Application No. 60/929,005, filed Jun. 7, 2007, each hereby expressly incorporated by reference in its entirety and each assigned to the assignee hereof.

### BACKGROUND OF THE INVENTION

### Technical Field of the Invention

The invention relates to a fragrancing composition comprising, in a cosmetically acceptable medium:

a) at least 2% by weight of a fragrancing substance, relative to the total weight of the composition;

b) at least one hydroxyaminobenzophenone screen A of specific formula (I) that will be defined below in detail;

c) at least one cinnamate screen B;

d) at least one compound C selected from among:

(i) a piperidinol compound of specific formula (II) that will be defined below in detail;

(ii) at least one benzotriazole compound of specific formula (III) that will be defined below in detail;

(iii) a UV screen of the dibenzoylmethane type.

It is known that a fragrance is the combination of various odorous substances which evaporate at different times. Each fragrance exhibits what is termed a “top note” which is the odor that diffuses first when the fragrance is applied or on opening the receptacle containing it, a “heart or middle note” which corresponds to the complete fragrance (emitted for several hours after the “top note”) and a “bottom note” which is the most persistent odor (emitted for several hours after the “heart note”). The persistence of the bottom note corresponds to how long the fragrance lingers.

Human beings have always wanted to fragrance themselves and the objects surrounding them or the places surrounding them, both in order to mask strong and/or unpleasant odors and to provide a pleasant odor.

It is common practice to incorporate some fragrance into a certain number of products or compositions, in particular cosmetic or dermatological products or compositions, such as splashes, toilet water, eau de parfum, aftershave lotions and skin care water.

A fragrance or a fragrancing composition should have a pleasant odor and a color that are stable over a certain period of time corresponding to the time during which it is used by the consumer. They should withstand various attacks such as the light and temperature differences.

A screening system and/or an antioxidant system are generally added. However, certain screening systems are ineffective and/or have a tendency to denature the desired color and/or the desired fragrance.

Most fragrancing products comprise stabilizers such as UV-A screens or UV-B screens and antioxidants.

The role of the stabilizers is to keep the organoleptic properties of the products stable with respect to various attacks such as UV radiation and heat shocks. Stabilizing agents are known, from the prior art, to improve the resistance of these products with respect to these attacks.

The combination of diethylamino hydroxybenzoylhexylbenzoate and ethylhexyl methoxycinnamate, marketed under the trademark Uvinul A+B, is known, from WO 2005/123013, to stabilize the color in fragrancing compositions.

The use of Tinogard AS or “bumetrizole” for preventing photolytic degradation of cosmetic products, including fragrances, is also known from WO 2000/25730.

The use of Tinogard Q or “tris(tetramethylhydroxypiperidinol)” for stabilizing fragrances, including toilet water, is also known from WO 2003/103622.

WO 2005/042828 mentions the combination of Tinogard Q with specific screens for the purpose of stabilizing fragrances and preventing the light-induced degradation of toilet water.

Unfortunately, the UV screens or the stabilizers recommended by the patent applications of the prior art are not entirely satisfactory, in particular for delicate fragrances rich in natural substances. Modifications of the organoleptic properties, such as in particular those of the odor and of the color over time (judged by sensory analysis such as the “sniff test” for the odor and a test of visualization to the naked eye or by means of a spectrometer in order to quantify the changes in color according to various parameters of color change, according to exposure to various types of lightness) are most commonly observed. In particular, among visual modifications, yellowing and reddening linked to the degradation of chemical substances readily appear. Modifications of the clarity of liquids can also be seen, when said liquids are originally transparent, such as flocculations and precipitates of substances or cloudiness that alters the appearance of the products and that may be detrimental to the effectiveness of pumps, when the product is packaged as a spray.

Thus, there remains therefore the need to search for new fragrancing products which do not exhibit the drawbacks of the prior art products, and in particular the need for fragrancing products whose organoleptic properties, such as odor and color, remain stable over time and under the effects of the light or temperature differences.

The assignee hereof has discovered, surprisingly, that this objective can be achieved by using a fragrancing composition comprising, in a cosmetically acceptable medium:

a) at least 2% by weight of a fragrancing substance, relative to the total weight of the composition;

b) at least one hydroxyaminobenzophenone screen A of specific formula (I) that will be defined below in detail;

c) at least one cinnamate screen B;

d) at least one compound C selected from among:

(i) a piperidinol compound of specific formula (II) that will be defined below in detail;

(ii) at least one benzotriazole compound of specific formula (III) that will be defined below in detail;

(iii) a UV screen of the dibenzoylmethane type.

This discovery forms the basis of the invention.

The invention also relates to the cosmetic use of the combination of at least one hydroxyaminobenzophenone screen A of specific formula (I) that will be defined below in detail, at least one cinnamate screen B and at least one compound C selected from among:

(i) a piperidinol compound of specific formula (II) that will be defined below in detail;

(ii) at least one benzotriazole compound of specific formula (III) that will be defined below in detail;

(iii) a UV screen of the dibenzoylmethane type;

in a fragranced composition comprising, in a cosmetically acceptable medium, at least 2% by weight of a fragrancing substance, as an agent for stabilizing the organoleptic properties, and in particular the color and/or the odor, of said composition with respect to outside attacks such as the light or temperature differences.

A subject of the invention is also a cosmetic process for fragrancing human keratin fibers, and in particular the skin, the lips, the hair, the scalp and the nails, comprising the application of the composition as defined above to the keratin materials.

The term “fragrancing composition” means any composition which leaves a fragrance after application to the keratin materials.

The term “fragrancing substance” means any fragrance or aroma capable of fragrancing the skin and human keratin materials, in general, comprising the skin, the hair, the scalp, the lips and the nails.

The amount of fragrancing substance(s) will more preferably be from 3% to 50% by weight, better still from 5% to 30%, even better still from 10% to 20% by weight, relative to the total weight of the composition.

The term “cosmetically acceptable medium” means, in the composition of the invention, a non-toxic medium that can be applied to human keratin materials comprising the skin, the face, the lips, the nails, the hair and the scalp.

Fragrancing substances that may be used in the composition of the invention include fragrances and aromas of natural or synthetic origin, and mixtures thereof. As fragrances and aromas of natural origin, mention may, for example, be made of extracts of flowers (lily, lavender, rose, jasmine, ylang-ylang), of stems and of leaves (patchouli, geranium, bitter leaf), of fruits (coriander, anise, cumin, juniper), of fruit peel (bergamot, lemon, orange), of roots (angelica, celery, cardamom, iris, sweet flag), of wood (pinewood, sandalwood, lignum vitae, pink cedar), of herbs and grasses (tarragon, lemongrass, sage, thyme), of needles and branches (spruce, fir, pine, dwarf pine), of resins and of balsams (galbanum, gum elemi, gum benzoin, myrrh, frankincense, opopanax).

As fragrancing substance of synthetic origin, mention may, for example, be made of compounds of ester, ether, aldehyde, ketone, aromatic alcohol and hydrocarbon type.

As esters, mention may in particular be made of benzyl acetate, benzyl benzoate, phenoxyethyl isobutyrate, p-tert-butylcyclohexyl acetate, citronellyl acetate, citronellyl formate, geranyl acetate, linalyl acetate, dimethylbenzylcarbinyl acetate, phenylethyl acetate, linalyl benzoate, benzyl formate, ethylmethylphenyl glycinate, alkylcyclohexyl propionate, styralyl propionate and benzyl salicylate.

As ethers, mention may be made of benzyl ethyl ether.

As aldehydes, mention may, for example, be made of linear alkanals having from 8 to 18 carbon atoms, citral, citronellal, cintronellyloxyacetaldehyde, cyclamenaldehyde, hydroxycitronellal, lilial and bourgeonal.

As ketones, mention may, for example, be made of ionones such as alpha-isomethylionone and methyl cedryl ketone.

As aromatic, and in particular terpenic, alcohols, mention may be made of anethol, citronellol, eugenol, isoeugenol, geraniol, linalol, phenylethyl alcohol and terpineol.

As hydrocarbons, mention may in particular be made of terpenes. These compounds are often in the form of a mixture of two or more of these odorous substances.

Moreover, it is also possible to use essential oils, and components of aromas, such as, for example, sage, chamomile, clove, Melissa, mint, cinnamon leaf, lime blossom, juniper, vetiver, frankincense, galbanum, labolanum and lavandin essences.

As fragrancing substance, use is preferably made, alone or as a mixture, of essence of bergamot, dihydromyrcenol, lilial, lyral, citronellol, phenylethyl alcohol, alpha-hexylcinnamaldehyde, geraniol, benzylacetone, cyclamenaldehyde, linalol, ambroxan, indole, hedione, sandelice, lemon, mandarin and orange essences, allylamine glycolate, cyclovertal, essence of lavandin, essence of sage, betadamascone, essence of geranium, cyclohexyl salicylate, phenylacetic acid, geranyl acetate, benzyl acetate and rose oxide.

According to a preferred embodiment of the invention, a mixture of various fragrancing substances which jointly generate a note that is pleasant to the user. Among the known olfactory notes, mention may, for example, be made of hesperide fragrances, aromatics, floral fragrances, musks, fruity fragrances, spicy fragrances, oriental fragrances, oceanic fragrances, aquatic notes, chypre fragrances, woody fragrances, Fougeres fragrances, and mixtures thereof.

The hydroxyaminobenzophenone compounds in accordance with the invention correspond to general formula (I) below:

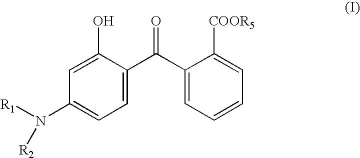

in which:

R₁ and R₂, which may be identical or different, denote a hydrogen atom, a C₁-C₂₀ alkyl radical, a C₃-C₁₀ cycloalkyl or a C₃-C₁₀ cycloalkenyl or form, with the nitrogen atom to which they are attached, a 5- or 6-membered ring;

R₃ is a C₁-C₂₀ alkyl radical.

As C₁-C₂₀ alkyl radicals, mention may, for example, be made of: methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl.

As C₃-C₁₀ cycloalkyl radicals, mention may, for example, be made of: cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentylcyclopropyl, 1-methyl-1-butylcyclopropyl, 1,2-dimethylcyclopropyl, 1-methyl-2-ethylcyclopropyl, cyclooctyl, cyclononyl or cyclodecyl.

As C₃-C₁₀ cycloalkenyl radicals having one or more double bonds, mention may be made of: cyclopropenyl, cyclobutenyl, cyclopentenyl, cyclopentadienyl, cyclohexenyl, 1,3-cyclohexadienyl, 1,4-cyclohexadienyl, cycloheptenyl, cycloheptatrienyl, cyclooctenyl, 1,5-cyclooctadienyl, cyclooctatetraenyl, cyclononenyl or cyclodecenyl.

As examples of a 5- or 6-membered ring formed by the radicals R₁ and R₂ with the nitrogen atom, mention may in particular be made of pyrrolidine or piperidine.

A compound A of formula (I) that is most particularly preferred is n-hexyl 2-(4-diethylamino-2-hydroxybenzoyl)benzoate of formula (A):

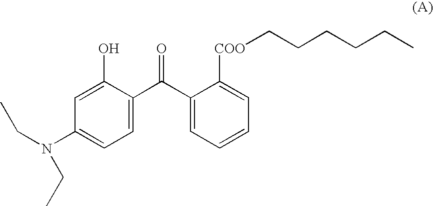

such as the product marketed under the trademark “Uvinul A+” by BASF.

The compounds A of formula (I) as defined above are known in themselves and the structures and syntheses thereof are described in EP-A-1-046,391, EP-1-133,980 and DE 100 12 408.

Preferably, the hydroxyaminobenzophenone compound A of formula (I) is present in the compositions according to the invention at a content ranging from 0.01% to 1.5% by weight, and more particularly from 0.1% to 0.8% by weight, relative to the total weight of the composition.

Among the cinnamate screens B in accordance with the invention, mention may in particular be made, in a non-limiting manner, of:

Ethylhexyl methoxycinnamate marketed in particular under the trademark “Parsol MCX” by Hoffmann La Roche,

isopropyl methoxycinnamate,

isoamyl methoxycinnamate marketed under the trademark “Neo Heliopan E 1000” by Haarmann and Reimer,

cinoxate,

DEA methoxycinnamate,

diisopropyl methylcinnamate, glyceryl ethylhexanoate dimethoxycinnamate.

Among the cinnamate screens B mentioned above, use will more particularly be made of ethylhexyl methoxycinnamate marketed under the trademark “Parsol MCX” by DSM; this screen corresponds to formula (B) below:

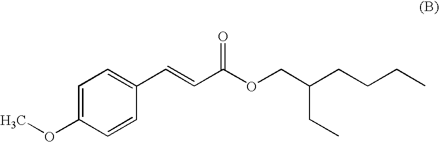

Preferably, the cinnamate compound B is present in the compositions according to the invention at a content ranging from 0.01% to 1.5% by weight, and more particularly from 0.1% to 0.8% by weight, relative to the total weight of the composition.

According to a specific form of the invention, use will be made of the n-hexyl 2-(4-diethylamino-2-hydroxybenzoyl)benzoate/ethylhexyl methoxycinnamate combination in a ratio by weight of 35/65% by weight, such as the product marketed under the trademark “Uvinul A+B” by BASF.

This specific combination will be present in the compositions according to the invention at a content ranging from 0.01% to 2% by weight, and more particularly from 0.1% to 1% by weight, and even more particularly from 0.3% to 1% by weight, relative to the total weight of the composition.

The compound C in accordance with the invention is selected from among:

(i) a piperidinol compound of specific formula (II) that will be defined below in detail;

(ii) at least one benzotriazole compound of specific formula (III) that will be defined below in detail;

(iii) a dibenzoylmethane UV screen.

The piperidinol compounds in accordance with the invention correspond to formula (II) below:

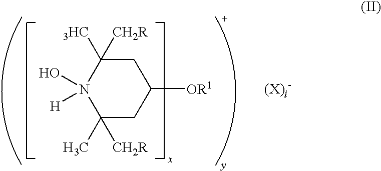

in which:

R is hydrogen or methyl;

x is 1 or 2;

1) when x is equal to 1:

R¹ is a hydrogen; a C₁-C₁₈ alkyl radical; a C₂-C₁₈ alkenyl radical; a propargyl radical; a glycidyl group; a C₂-C₅₀ alkyl radical interrupted with 1 to 20 oxygen atoms, said alkyl being substituted with 1 to 10 hydroxyl groups or else both interrupted with said oxygen atoms and substituted with said hydroxyl groups; a C₁-C₄ alkyl radical substituted with a carboxyl group or a —COOZ group where Z is hydrogen, a C₁-C₄ alkyl, phenyl, a C₁-C₄ alkyl substituted with a (COO⁻)ₚ M^{p+} group where p is an integer from 1 to 3 and M a metal ion of groups 1, 2 and 3 of the Periodic Table or Zn, Cu, Ni or Co or else M is an N^{p+} (R²)₄ group where R² is a C₁-C₈ alkyl or a benzyl;

2) when x is 2:

R¹ is a C₁-C₁₂ alkylene radical; a C₄-C₁₂ alkenylene radical; a xylylene group; a C₁-C₅₀ alkylene radical interrupted with 1 to 20 oxygen atoms, said alkyl being substituted with 1 to 10 hydroxyl groups, or else both interrupted with said oxygen atoms and substituted with said hydroxyl groups;

X is an organic or inorganic anion;

the total cation charge y being equal to the total anion charge j.

Among the anions X, mention may be made of phosphate, phosphonate, carbonate, bicarbonate, nitrate, chloride, bromide, bisulfite, sulfite, bisulfate, sulfate, borate, formate, acetate, benzoate, citrate, oxalate, tartrate, acrylate, polyacrylate, fumarate, maleate, itaconate, glycolate, gluconate, malate, mandelate, tiglate, ascorbate, polymethacrylate, a nitrilotriacetic acid carboxylate, hydroxyethylenediaminetriacetic acid, ethylenediaminetetraacetic acid, diethylenediaminepentaacetic acid, diethylenetriaminepentamethylene phosphonate, an alkyl sulfonate or an aryl sulfonate.

The piperidinol compounds of formula (II) are known in themselves and described in WO 03/103622.

Use will in particular be made of the compounds of formula (II) for which R and R¹ denote hydrogen, x=1 and X is the citrate anion, and even more particularly the tris(tetramethylhydroxypiperidinol) citrate compound having the structure:

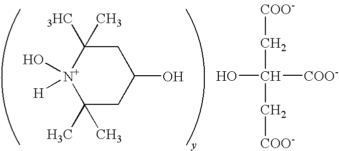

with y=3, such as the commercial product marketed under the trademark Tinoguard Q or Tinoguard S-FX by Ciba-Geigy.

The benzotriazole compounds in accordance with the invention correspond to the formula below:

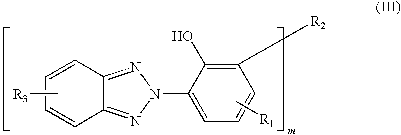

in which:

R₁ is a C₁-C₁₂ alkyl radical, a C₁-C₅ alkoxy radical, a C₁-C₅ alkoxycarbonyl, a C₅-C₇ cycloalkyl, a C₆-C₁₀ aryl, and aralkyl radical, an SO₃M group, or a radical of formula (IIIa)

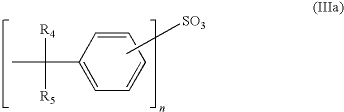

R₄ and R₅, which may be identical or different, denote a C₁-C₅ alkyl or a hydrogen;

R₃ is hydrogen, a C₁-C₁₂ alkyl radical, a C₁-C₅ alkoxy radical, a halogen, preferably Cl, or a hydroxyl group;

M is an alkali metal or alkaline-earth metal cation or an ammonium cation, preferably Na⁺;

m is 1 or 2;

n is 0 or 1;

if m=1, then R₂ is hydrogen, a C₁-C₁₂ alkyl group which is unsubstituted or substituted with a phenyl; or a C₆-C₁₀ aryl; if m=2, then R₂ is a direct linkage or a —(CH₂)ₚ— radical with p being an integer from 1 to 3.

The alkyl radicals are linear or branched and are selected, for example, from methyl, ethyl, n-propyl, isopropyl, n-butyl, sec-butyl, tert-butyl, amyl, isoamyl, heptyl, octyl, isooctyl, nonyl, decyl and dodecyl.

As C₁-C₅ alkoxy radicals, mention may be made of: methoxy, ethoxy, n-propoxy, n-butoxy, n-pentoxy, 1-methylpropoxy, 3-methylbutoxy or 2,2-dimethylpropoxy.

The C₅-C₇ cycloalkyls are, for example, cyclopentyl, cycloheptyl or, preferably, cyclohexyl.

The alkylphenyls are, for example, phenylpropyl, phenylethyl and, preferably, benzyl.

The benzotriazole compounds of formula (III) are known in themselves and their structures are described in WO 00/25730.

Among the compounds of formula (III), mention may in particular be made of the following compounds:

(i) sodium benzotriazolyl butylphenol sulfonate of formula (I)

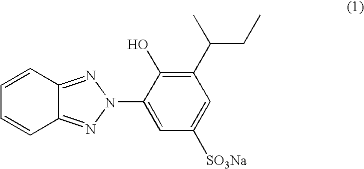

such as the product marketed under the trademark “Tinoguard HS” by Ciba-Geigy.
(ii) benzotriazolyl dodecyl p-cresol of formula (2):

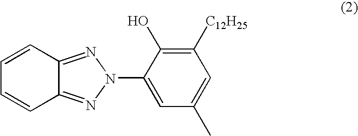

such as the product marketed under the trademark “Tinoguard TL” by Ciba-Geigy.
(iii) benzotriazolyl butylphenol sulfonate of formula (3):

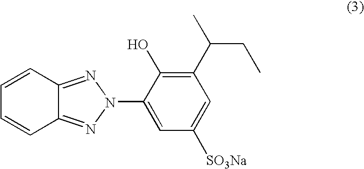

such as the product marketed under the trademark “Cibafast H Liquid” by Ciba-Geigy.
(iv) bumetrizole of formula (4):

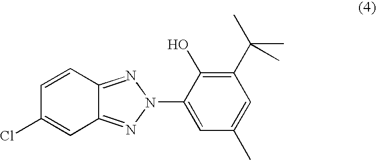

such as the product marketed under the trademark “Tinoguard AS” by Ciba-Geigy.

The bumetrizole compound of formula (4) will more particularly be used.

The compound(s) C of the piperidinol type and also those of the benzotriazole type, in accordance with the invention, are preferably present in the compositions according to the invention at a content ranging from 0.01% to 0.1% of the final composition, more preferably from 0.01% to 0.05% by weight, and even more particularly from 0.01% to 0.03% by weight, relative to the total weight of the composition.

Among the dibenzoylmethane screens in accordance with the invention, mention may in particular be made, in a non-limiting manner, of:
2-methyldibenzoylmethane,
4-methyldibenzoylmethane,
4-isopropyldibenzoyl methane,
4-tert-butyldibenzoyl methane,
2,4-dimethyldibenzoylmethane,
2,5-dimethyldibenzoylmethane,
4,4′-diisopropyldibenzoylmethane,
4,4′-dimethoxydibenzoyl methane,
4-tert-butyl-4′-methoxydibenzoyl methane,
2-methyl-5-isopropyl-4′-methoxydibenzoyl methane,
2-methyl-5-tert-butyl-4′-methoxydibenzoyl methane,
2,4-dimethyl-4′-methoxydibenzoyl methane,
2,6-dimethyl-4-tert-butyl-4′-methoxydibenzoyl methane.

Among the dibenzoylmethanes mentioned above, use will more particularly be made of 4-(tert-butyl)-4′-methoxydibenzoylmethane or butyl methoxy dibenzoylmethane, marketed under the trademark “Parsol 1789” by DSM; this screen corresponds to formula (IV) below:

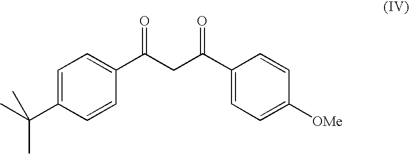

The dibenzoylmethane screen(s) in accordance with the invention is (are) preferably present in the compositions according to the invention at a content ranging from 0.01% to 1.5% by weight, preferably from 0.1% to 0.5% by weight, and more particularly from 0.1% to 0.8% by weight, relative to the total weight of the composition.

According to a specific form of the invention, the fragrancing composition contains, in a cosmetically acceptable medium:

a) at least 2% by weight of a fragrancing substance, relative to the total weight of the composition;

b) at least one hydroxyaminobenzophenone screen A of formula (I) as defined above;

c) at least one cinnamate screen B as defined above;

d) at least one dibenzoylmethane screen C as defined above, and

e) at least one screen D of the salicylate type.

Among the screens D of the salicylate type in accordance with the invention, mention may in particular be made, for example, of:

Homosalate marketed under the trademark “Eusolex HMS” by Rona/EM Industries,

Ethylhexyl salicylate marketed under the trademark “Neo Heliopan OS” by Symrise,

Dipropylene glycol salicylate marketed under the trademark “Dipsal” by Scher,

TEA salicylate, marketed under the trademark “Neo Helipoan TS” by Symrise.

Among the salicylates D mentioned above, use will most particularly be made of the ethylhexyl salicylate marketed under the trademark “Neo Heliopan OS” by Symrise of formula (V) below:

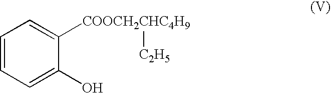

The screen(s) D of the salicylate type in accordance with the invention is (are) preferably present in the compositions according to the invention at a content ranging from 0.01% to 1.5% by weight, preferably from 0.1% to 0.5% by weight, and more particularly from 0.1% to 0.8% by weight, relative to the total weight of the composition.

According to a specific form of the invention, the cosmetically acceptable medium in accordance with the present invention contains at least one volatile alcohol and/or one volatile silicone oil and, optionally, water. Preferably, this medium of the composition contains water in an amount preferably ranging from 0.01% to 50%, and more preferably from 0.5% to 25% by weight, relative to the total weight of the composition.

The term “volatile alcohol” means any compound comprising at least one alcohol function having a vapor pressure at 20° C. of greater than 17.5 mm of mercury.

For the purpose of the invention, the term “oil” means a water-insoluble fatty substance that is liquid at ambient temperature and atmospheric pressure.

For the purpose of the invention, the term “volatile silicone oil” means any silicone-based compound capable of evaporating on contact with the skin or with the keratin fiber in less than one hour, at ambient temperature and atmospheric pressure. The volatile compound is a volatile cosmetic compound which is liquid at ambient temperature and which has in particular a non-zero vapor pressure at ambient temperature and atmospheric pressure, in particular which has a vapor pressure ranging from 0.13 Pa to 40,000 Pa (10⁻³ to 300 mmHg), in particular ranging from 1.3 Pa to 13,000 Pa (0.01 to 100 mmHg), and more particularly ranging from 1.3 Pa to 1,300 Pa (0.01 to 10 mmHg).

The volatile alcohols in accordance with the present invention are preferably selected from among C₁-C₅ lower monoalcohols, and can be selected from among methanol, ethanol, propanol, isopropanol, n-butanol, isobutanol and t-butanol, and more particularly ethanol.

The volatile alcohol(s) is (are) preferably present in amounts ranging from 40% to 80%, and more preferably in amounts ranging from 55% to 80% by weight, relative to the total weight of the composition.

As volatile silicone oils, mention may, for example, be made of linear or cyclic volatile silicone oils, in particular those having a viscosity ≦6 centistokes (6×10⁻⁶ m²/s) and having in particular from 2 to 10 silicon atoms, these silicones optionally comprising alkyl or alkoxy groups having from 1 to 22 carbon atoms. As a volatile silicone oil that can be used in the invention, mention may in particular be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane and mixtures thereof.

The volatile silicone oil(s) is (are) preferably present at from 40% to 98.5%, preferably in concentrations ranging from 10% to 80% by weight, relative to the total weight of the composition.

The composition of the invention may also comprise any additive normally used in the fragrance field, selected in particular from antioxidants, cosmetic or dermatological active agents such as, for example, emollients or demulcents, for instance sweet almond oil, apricot kernel oil, moisturizers such as glycerol, calmatives such as a-bisabolol, allantoin, aloe vera; vitamins, essential fatty acids, insect repellants, propellants, peptizers, dyestuffs, pearlescent agents and flakes, and mixtures thereof. The composition of the invention may also contain adjuvants commonplace in the cosmetics or dermatological field, such as hydrophilic or lipophilic gelling agents, preservatives (for example, phenoxyethanol and parabens), solvents, fillers or bactericides. The amounts of these various adjuvants are those conventionally used in the field under consideration, and are, for example, from 0.01% to 20% of the total weight of the composition.

Among the antioxidants, mention may, for example, be made of BHA (tert-butyl-4-hydroxyanisole), BHT (2,6-di-tert-butyl-p-cresol), and tocopherols, for instance vitamin E and its derivatives such as tocopheryl acetate.

The composition according to the invention may in particular comprise at least one dyestuff such as pulverulent dyestuffs, liposoluble dyes or water-soluble dyes.

The pulverulent dyestuffs can be selected from among pigments and pearlescent agents.

The pigments may be white or colored, inorganic and/or organic, and coated or uncoated. Among the inorganic pigments, mention may be made of optionally surface-treated titanium dioxide, zirconium oxides, zinc oxides or cerium oxides, and also iron oxides or chromium oxides, manganese violet, ultramarine blue, chromium hydrate and ferric blue. Among the organic pigments, mention may be made of carbon black, pigments of D and C type, and lakes based on cochineal carmine, on barium, on strontium, on calcium or on aluminum.

The pearlescent agents may be selected from among white pearlescent pigments such as mica coated with titanium or with bismuth oxychloride, colored pearlescent pigments such as titanium mica with iron oxides, titanium mica with in particular ferric blue or with chromium oxide, titanium mica with an organic pigment of the abovementioned type, and also pearlescent pigments based on bismuth oxychloride.

The soluble dyes are, for example: caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, cochineal carmine (CI-15850, CI 75470), Ext. Violet 2, Red 6-7, Ferric Ferrocyanide, Ultramarines, Acid Yellow 3/Yellow 10, Acid Blue 3, Yellow 10.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

The invention applies not only to fragrancing products, but also to care products and treatment products for the body, the skin, the face, the lips and the nails, including the hair and the scalp, containing an odorous substance. The composition according to the invention may thus constitute a fragrancing, care or treatment composition for keratin materials, and in particular be in the form of a splash, toilet water, eau de parfum, and aftershave lotion, a care water, a silicone or hydrosilicone care oil, or hygiene products such as shower gels, bath products, shampoos or scrubs. It may also be in the form of a two-phase or three-phase fragrancing lotion (toilet water phase/hydrocarbon-based oil and/or silicone oil and/or fluoro oil phase).

A subject of the invention is also a cosmetic process for fragrancing human keratin materials, and in particular the skin, the lips and the appendages, comprising the application to the keratin materials of the composition as defined above.

The composition according to the invention can be produced by known methods, generally used in the fragranced composition field.

The compositions according to the invention can be packaged in the form of bottles. They can also be applied in the form of fine particles by means of pressurization devices. The devices in accordance with the invention are well known to those skilled in the art and comprise non-aerosol pump-dispensers or “atomizers”, aerosol containers comprising a propellant and also aerosol pump-dispensers using compressed air as propellant. The latter are described in U.S. Pat. Nos. 4,077,441 and 4,850,517 (forming an integral part of the content of the description).

The compositions packaged in an aerosol, in accordance with the invention, generally contain conventional propellants such as, for example, the hydrofluorinated compounds dichlorodifluoromethane or difluoroethane, dimethyl ether, isobutane, n-butane, propane or trichlorofluoromethane.

The fragrancing compositions according to the invention are in all the galenical forms conventionally used for topical application, and in particular in the form of aqueous or aqueous-alcoholic solutions, oil-in-water (O/W) or water-in-oil (W/O) or multiple (triple: W/O/W or O/W/O) emulsions, aqueous gels, dehydrated anhydrous products such as loose or compact fragrancing powders, or dispersions of an oily phase in an aqueous phase by means of lipid vesicles of ionic type (liposomes) and/or non-ionic type. These compositions are prepared according to the usual methods.

In addition, the compositions according to the invention may be more or less fluid and may have the appearance of a cream, an ointment, a milk, a lotion, a serum, a paste or a mousse. They may also be in solid form, and for example in the form of a stick.

When the composition according to the invention comprises an oily phase, the latter preferably contains at least one oil, in particular one physiogically acceptable oil. It may also contain other fatty substances.

As oils that can be used in the composition of the invention, mention may, for example, be made of:

hydrocarbon-based oils of animal origin, such as perhydrosqualene;

hydrocarbon-based oils of plant origin, such as liquid triglycerides of fatty acids having from 4 to 10 carbon atoms, for instance heptanoic acid or octanoic acid triglycerides, or alternatively, for example, sunflower oil, maize oil, soybean oil, marrow oil, grapeseed oil, sesame oil, hazelnut oil, apricot oil, macadamia oil, arara oil, castor oil, avocado oil, caprylic/capric acid triglycerides such as those marketed by Stearineries Dubois or those marketed under the trademarks Miglyol 810, 812 and 818 by Dynamit Nobel, jojoba oil, shay butter oil;

synthetic esters and ethers, in particular of fatty acids, such as oils of formulae R¹COOR² and R₁OR₂ in which R¹ is the residue of a fatty acid having from 8 to 29 carbon atoms, and R² is a branched or non-branched hydrocarbon-based chain having from 3 to 30 carbon atoms, for instance Purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, fatty alcohol heptanoates, octanoates or decanoates; polyol esters, for instance propylene glycol dioctanoate, neopentyl glycol diheptanoate and diethylene glycol diisononanoate; and pentaerythritol esters such as pentaerythrityl tetraisostearate;

linear or branched hydrocarbons of mineral or synthetic origin, such as volatile or non-volatile liquid paraffins, and derivatives thereof, petroleum jelly, polydecenes, or hydrogenated polyisobutene such as Parleam oil;

fatty alcohols having from 8 to 26 carbon atoms, such as cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluoro oils such as those described in JP-A-2-295912;

volatile or non-volatile silicone oils, such as polydimethylsiloxanes (PDMSs), comprising a linear or cyclic silicone chain, which are liquid or pasty at ambient temperature, in particular cyclopolydimethylsiloxanes (cyclomethicones) such as cyclohexasiloxane; polydimethylsiloxanes comprising alkyl, alkoxy or phenyl groups which are pendent or at the end of a silicone carbon chain; phenyl silicones such as phenyl trimethicones, phenyl dimethicones, phenyltrimethylsiloxydiphenylsiloxanes, diphenyl dimethicones, diphenylmethyldiphenyltrisiloxanes, 2-phenylethyltrimethyl siloxysilicates, and polymethylphenylsiloxanes; and mixtures thereof.

In the list of oils mentioned above, the term “hydrocarbon-based oil” means any oil comprising predominantly carbon and hydrogen atoms and, optionally, ester, ether, fluoro, carboxylic acid and/or alcohol groups.

The other fatty substances that may be present in the oily phase are, for example, fatty acids having from 8 to 30 carbon atoms, such as stearic acid, lauric acid, palmitic acid and oleic acid; waxes such as lanolin, beeswax, carnauba wax or candelilla wax, paraffin waxes, lignite waxes or microcrystalline waxes, ceresine or ozokerite, synthetic waxes such as polyethylene waxes, Fischer-Tropsch waxes; silicone resins such as trifluoromethyl(C₁-C₄)alkyl dimethicone and trifluoropropyl dimethicone; and silicone elastomers such as the products marketed under the trademarks “KSG” by Shin-Etsu, under the trademarks “Trefil”, “BY29” or “EPSX” by Dow Corning or under the trademark “Gransil” by Grant Industries.

These fatty substances may be selected in a varied manner by those skilled in the art in order to prepare a composition having the desired properties, for example of consistency or of texture.

The emulsions generally contain at least one emulsifier selected from among amphoteric, anionic, cationic or non-ionic emulsifiers, used alone or as a mixture. The emulsifiers are selected appropriately according to the continuous phase of the emulsion to be obtained (W/O or O/W). When the emulsion is a multiple emulsion, it generally comprises an emulsifier in the primary emulsion and an emulsifier in the external phase into which the primary emulsion is introduced.

As emulsifiers that can be used for the preparation of the W/O emulsions, mention may, for example, be made of alkyl esters or ethers of sorbitan, of glycerol or of sugars; silicone surfactants, for instance dimethicone copolyols such as the mixture of cyclomethicone and dimethicone copolyol marketed under the trademarks DC 5225 C and DC 3225 C by Dow Corning, and for instance alkyl dimethicone copolyols such as the lauryl methicone copolyol marketed under the trademark “Dow Corning 5200 Formulation Aid” by Dow Corning, the cetyl dimethicone copolyol marketed under the trademark Abil EM 90R by Goldschmidt and the mixture of polyglyceryl-4 isostearate/cetyl dimethicone copolyol/hexyl laurate marketed under the trademark Abil WE 09R by Goldschmidt. It is also possible to add thereto one or more coemulsifiers which, advantageously, can be selected from among the group comprising esters of a branched-chain fatty acid and of a polyol, and in particular esters of a branched-chain fatty acid and of glycerol and/or of sorbitan, and for example polyglyceryl isostearate, such as the product marketed under the trademark Isolan GI 34 by Goldschmidt, sorbitan isostearate, such as the product marketed under the trademark Arlacel 987 by ICI, sorbitan glycerol isostearate, such as the product marketed under the trademark Arlacel 986 by ICI, and mixtures thereof.

As emulsifiers that can be used for the preparation of the O/W emulsions, mention may, for example, be made of non-ionic emulsifiers such as oxyalkylenated (more particularly polyoxyethylenated) polyol fatty acid esters, and for example polyethylene glycol stearates such as PEG-100 stearate, PEG-50 stearate and PEG-40 stearate; and mixtures thereof such as the mixture of glyceryl monostearate and polyethylene glycol stearate (100 EO) marketed under the trademark Simulsol 165 by Seppic; oxyalkylenated sorbitan fatty acid esters comprising, for example, from 20 to 100 EO, and for example those marketed under the trademarks Tween 20 or Tween 60 by Ubiqema; oxyalkylenated (oxyethylenated and/or oxypropylenated) fatty alcohol ethers; sugar esters such as sucrose stearate; and mixtures thereof, for instance the mixture of glyceryl stearate and PEG-100 stearate, marketed under the trademark Arlacel 165 by Uniqema.

It is possible to add, to these emulsifiers, coemulsifiers such as, for example, fatty alcohols having from 8 to 26 carbon atoms, for instance cetyl alcohol, stearyl alcohol and the mixture thereof (cetearyl alcohol), octyldodecanol, 2-butyloctanol, 2-hexyldecanol, 2-undecylpentadecanol or oleyl alcohol.

It is also possible to prepare emulsions without emulsifying surfactants or containing less than 0.5% thereof relative to the total weight of the composition, using appropriate compounds, for example polymers having emulsifying properties, such as the polymers marketed under the trademarks Carbopol 1342 and Pemulen by Noveon; or optionally crosslinked and/or neutralized 2-acrylamido-2-methylpropanesulfonic acid polymers and copolymers, such as the poly(2-acrylamido-2-methylpropanesulfonic acid) marketed by Clariant under the trademark “Hostacerin AMPS” (CTFA name: ammonium polyacryldimethyltauramide) or such as the polymer in emulsion marketed under the trademark Sepigel 305 by Seppic (INCI name: polyacrylamide/C13-C14 isoparaffin/laureth-7); particles of ionic or non-ionic polymers, more particularly particles of anionic polymer, such as in particular isophthalic acid or sulfoisophthalic acid polymers, and in particular the phthalate/sulfoisophthalate/glycol copolymers (for example, diethylene glycol/phthalate/isophthalate/1,4-cyclohexanedimethanol (INCI name: Diglycol/CHDM/Isophthalates/SIP Copolymer) marketed under the trademark Eastman AQ polymer (AQ35S, AQ38S, AQ55S, AQ48 Ultra) by Eastman Chemical.

It is also possible to prepare emulsions without emulsifiers, stabilized with silicone particles or particles of metal oxide such as TiO₂ or the like.

As fillers which may be used in the composition of the invention, mention may, for example, be made, in addition to the pigments, of silica powder; talc; polyamide particles, and in particular those marketed under the trademark Orgasol by Atochem; polyethylene powders; powders of natural organic materials such as starch powders, in particular crosslinked or non-crosslinked maize, wheat or rice starch powders, such as the powders of starch crosslinked with octenylsuccinate anhydride, marketed under the trademark Dry-Flo by National Starch; acrylic copolymer-based microspheres, such as those made of ethylene glycol dimethacrylate/lauryl methacrylate copolymer, marketed by Dow Corning under the trademark Polytrap; expanded powders such as hollow microspheres, and in particular the microspheres marketed under the trademark Expancel by Kemanord Plast or under the trademark Micropearl F 80 ED by Matsumoto; silicone resin microbeads such as those marketed under the trademark Tospearl by Toshiba Silicone; and mixtures thereof. These fillers may be present in amounts ranging from 0 to 20% by weight, and preferably from 1% to 10% by weight, relative to the total weight of the composition.

The invention will now be described with reference to the following examples, given by way of non-limiting illustration. In these examples, unless otherwise indicated, the amounts are expressed as percentages by weight. The following fragranced formulations were prepared; the amounts are indicated as percentages by weight

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Comparative Odor Stability And Color Stability Tests

Principle:

The principle of these tests consists in exposing the test products to a radiant light source with a well-defined spectral distribution and a well-quantified energy output.

The light sources commonly used are xenon lamps emitting through:

a platinum-coated quartz filter which deflects infrared radiation and eliminates it from the top of the apparatus;

and a glass filter, which by absorbing short-wavelength UV radiation makes it possible to simulate the radiation received behind a display window.

Material:

A CPS Suntest apparatus is used:

The illumination provided by a xenon lamp from 300 nm to 800 nm is fixed at 765 W/m² (value set by the manufacturer);

Optical filtration is provided by a quartz filter with an IR coating and a special window glass.

The color of each bottle is observed with the naked eye before and after exposure to the light with a sun-test of 16 hours at ambient temperature.

A “sniff test” is also carried out on a panel of 5 individuals on the samples at TO and after 2 months at ambient temperature and at 45° C.

### Examples 1a, 1b and 1c

| Composition | 1a(*) | 1b(*) | 1c (invention) |
|---|---|---|---|
| Concentrate of fragrance x | 15% | 15% | 15% |
| Ethanol | 65% | 65% | 65% |
| Dyes | qs | qs | qs |
| 35/65 mixture of n-hexyl | 1 | 0% | 1 |
| 2-(4-diethylamino-2- | | | |
| hydroxybenzoyl)benzoate and | | | |
| ethylhexyl methoxycinnamate | | | |
| (Uvinul A + B) | | | |
| Tris | 0 | 0.03 | 0.03 |
| (tetramethylhydroxypiperidinol) | | | |
| citrate - (Tinoguard Q) | | | |
| Water | qs 100% | qs 100% | qs 100% |

(*)not part of the invention

Results:

Test 1a:

Results at T0: A fragrance with a golden yellow color and a pleasant odor is obtained.

16 h suntest result: degradation of the color=discoloration—

Observation 2 months 45° C.: very clear alteration of the odor, pungent odor, - - -

Test 1b:

Result at T0: A fragrance with a golden yellow color and a pleasant odor is obtained.

16 h suntest result: degradation of the color=discoloration—

Observation 2 months 45° C.: degradation of the odor—

Test 1c (According to the Invention):

Result at T0: A fragrance with a golden yellow color and a pleasant odor is obtained.

16 h suntest result: the initial color is maintained.

Observation 2 months 45° C.: no change in odor.

It is observed that composition 1c according to the invention, comprising the n-hexyl 2-(4-diethylamino-2-hydroxybenzoyl)benzoate/ethylhexyl methoxycinnamate/tris(tetramethylhydroxypiperidinol) combination has an odor and a color that are stable to light exposure and to storage at 45° C., unlike compositions 1a and 1b (not part of the invention).

### Examples 2a, 2b and 2c

| Composition | 2a(*) | 2b(*) | 2c (invention) |
|---|---|---|---|
| Concentrate of fragrance x | 15% | 15% | 15% |
| Ethanol | 65% | 65% | 65% |
| Dyes | qs | qs | qs |
| 35/65 mixture of n-hexyl | 1 | 0% | 1 |
| 2-(4-diethylamino-2- | | | |
| hydroxybenzoyl)benzoate and | | | |
| ethylhexyl methoxycinnamate | | | |
| (Uvinul A + B) | | | |
| Bumetrizole - compound | 0 | 0.03 | 0.03 |
| (4) (Tinoguard AS) | | | |
| Water | qs 100% | qs 100% | qs 100% |

(*)not part of the invention

Results:

Test 2a:

Result at T0: A fragrance with a golden yellow color and a pleasant odor is obtained.

16 h suntest result: degradation of the color=discoloration—

Observation 2 months 45° C.: very clear alteration of the odor, pungent odor, - - -

Test 2b:

Result at T0: A fragrance with a golden yellow color and a pleasant odor is obtained.

16 h suntest result: degradation of the color=discoloration—

Observation 2 months 45° C.: degradation of the odor—

Test 2c (According to the Invention):

Result at T0: A fragrance with a golden yellow color and a pleasant odor is obtained.

16 h suntest result: the initial color is maintained.

Observation 2 months 45° C.: no change in odor.

It is observed that composition 2c according to the invention, comprising the n-hexyl 2-(4-diethylamino-2-hydroxybenzoyl)benzoate/ethylhexyl methoxycinnamate/bumetrizole combination has an odor and a color that are stable to light exposure and to storage at 45° C., unlike compositions 2a and 2b (not part of the invention).

### Examples 3a, 3b, 3c and 3d

| Composition | 3a(*) | 3b(*) | 3c(*) | 1d (invention) |
|---|---|---|---|---|
| Concentrate of fragrance x | 15% | 15% | 15% | 15% |
| Ethanol | 65% | 65% | 65% | 65% |
| Dyes | qs | qs | qs | qs |
| n-hexyl 2-(4-diethylamino-2- | 1% | 0 | 0 | 0 |
| hydroxybenzoyl)benzoate | | | | |
| (Uvinul A+) | | | | |
| 35/65 mixture of n-hexyl 2-(4- | | 1% | 0 | 1 |
| diethylamino-2- | | | | |
| hydroxybenzoyl)benzoate and | | | | |
| ethylhexyl methoxycinnamate | | | | |
| (Uvinul A + B) | | | | |
| Butylmethoxydibenzoylmethane | 0 | 0 | 0.3% | 0.3 |
| (Parsol 1789) | | | | |
| Water | qs 100% | qs 100% | qs 100% | qs 100% |

(*)not part of the invention

Results:

Test 3a:

Result at T0: A fragrance with a golden yellow color and a pleasant odor is obtained.

16 h suntest result: degradation of the color=discoloration—

Observation 2 months 45° C.: very clear alteration of the odor, pungent odor, - - -

Test 3b:

Result at T0: A fragrance with a golden yellow color and a pleasant odor is obtained.

16 h suntest result: degradation of the color=discoloration—

Observation 2 months 45° C.: degradation of the odor—

Test 3c:

Result at T0: A fragrance with a golden yellow color and a pleasant odor is obtained.

16 h suntest result: degradation of the color=discoloration (- - -)

Observation 2 months 45° C.: very clear degradation of the odor (- - -)

Test 3d (According to the Invention):

Result at T0: A fragrance with a golden yellow color and a pleasant odor is obtained

16 h suntest result: the initial color is maintained.

Observation 2 months 45° C.: very small change in odor (-).

It is observed that composition 3d according to the invention, comprising the n-hexyl 2-(4-diethylamino-2-hydroxybenzoyl)benzoate/ethylhexyl methoxycinnamate/butylmethoxydibenzoylmethane combination has an odor and a color that are stable to light exposure and to storage at 45° C., unlike compositions 3a, 3b and 3c (not part of the invention).

### Examples 4a, 4b and 4c

| Composition | 4a(*) | 4b(*) | 4c (invention) |
|---|---|---|---|
| Concentrate of fragrance x | 15% | 15% | 15% |
| Ethanol | 65% | 65% | 65% |
| Dyes | qs | qs | qs |
| 35/65 mixture of n-hexyl 2-(4- | 0 | 1% | 0.6 |
| diethylamino-2- | | | |
| hydroxybenzoyl)benzoate and | | | |
| ethylhexyl methoxycinnamate | | | |
| (Uvinul A + B) | | | |
| Ethylhexyl salicylate (NEO | 0.3% | 0% | 0.1 |
| Heliopan OS) | | | |
| Butylmethoxydibenzoylmethane | 0% | 0% | 0.2 |
| (Parsol 1789) | | | |
| Water | qs 100% | qs 100% | qs 100% |

(*)not part of the invention

Results:

Test 4a:

Result at T0: A fragrance with a golden yellow color and a pleasant odor is obtained.

16 h suntest result: degradation of the color=discoloration—

Observation 2 months 45° C.: very clear alteration of the odor, pungent odor, - - -

Test 4b:

Result at T0: A fragrance with a golden yellow color and a pleasant odor is obtained.

16 h suntest result: degradation of the color=discoloration—

Observation 2 months 45° C.: degradation of the odor—

Test 4c (According to the Invention):

Result at T0: A fragrance with a golden yellow color and a pleasant odor is obtained.

16 h suntest result: the initial color is maintained.

Observation 2 months 45° C.: no change in odor.

It is observed that composition 4c according to the invention, comprising the n-hexyl 2-(4-diethylamino-2-hydroxybenzoyl)benzoate/ethylhexyl methoxycinnamate/butylmethoxydibenzoylmethane/ethylhexyl salicylate combination, has an odor and a color that are stable to light exposure and to storage at 45° C., unlike compositions 4a and 4b (not part of the invention).

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. Fragrancing composition comprising, in a cosmetically acceptable medium:
a) at least 2% by weight of a fragrancing substance, relative to the total weight of the composition;
b) at least one hydroxyaminobenzophenone screen A of formula (I) 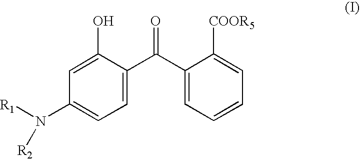
in which
R₁ and R₂, which may be identical or different, denote a hydrogen atom, a C₁-C₂₀ alkyl radical; a C₃-C₁₀ cycloalkyl or a C₃-C₁₀ cycloalkenyl or form, with the nitrogen atom to which they are attached, a 5- or 6-membered ring;
R₃ is a C₁-C₂₀ alkyl radical;
c) at least one cinnamate screen B;
d) at least one compound C selected from among:
(i) a piperidinol compound of formula (II) below: 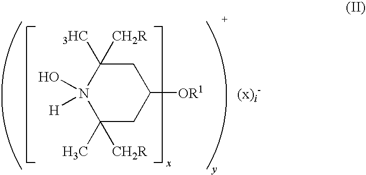
in which:
R is hydrogen or methyl;
x is 1 or 2;
1) when x is equal to 1:
R¹ is a hydrogen; a C₁-C₁₈ alkyl radical; a C₂-C₁₈ alkenyl radical; a propargyl radical; a glycidyl group; a C₂-C₅₀ alkyl radical interrupted with 1 to 20 oxygen atoms, said alkyl being substituted with 1 to 10 hydroxyl groups or else both interrupted with said oxygen atoms and substituted with said hydroxyl groups; a C₁-C₄ alkyl radical substituted with a carboxyl group or a —COOZ group where Z is hydrogen, a C₁-C₄ alkyl, phenyl, a C₁-C₄ alkyl substituted with a (COO⁻)ₚ M^{p+} group where p is an integer from 1 to 3 and M a metal ion of groups 1, 2 and 3 of the Periodic Table or Zn, Cu, Ni or Co or else M is an N^{p+} (R²)₄ group where R² is a C₁-C₈ alkyl or a benzyl;
2) when x is 2:
R¹ is a C₁-C₁₂ alkylene radical; a C₄-C₁₂ alkenylene radical; a xylylene group; a C₁-C₅₀ alkylene radical interrupted with 1 to 20 oxygen atoms, said alkyl being substituted with 1 to 10 hydroxyl groups, or else both interrupted with said oxygen atoms and substituted with said hydroxyl groups;
X is an organic or inorganic anion;
the total cation charge y being equal to the total anion charge j;
(ii) at least one benzotriazole compound of formula (III) 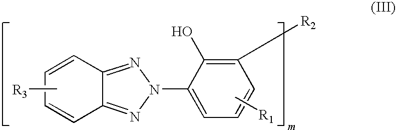
in which:
R₁ is a C₁-C₁₂ alkyl radical, a C₁-C₅ alkoxy radical, a C₁-C₅ alkoxycarbonyl, a C₅-C₇ cycloalkyl, a C₆-C₁₀ aryl, an aralkyl radical, an SO₃M group, or a radical of formula (IIIla) 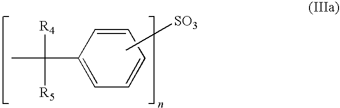
R₄ and R₅, which may be identical or different, denote a C₁-C₅ alkyl or a hydrogen;
R₃ is hydrogen, a C₁-C₁₂ alkyl radical, a C₁-C₅ alkoxy radical, a halogen, preferably Cl, or a hydroxyl group;
M is an alkali metal or alkaline-earth metal cation or an ammonium cation, preferably Na⁺;
m is 1 or 2;
n is 0 or 1;
if m=1, then R₂ is hydrogen, a C₁-C₁₂ alkyl group which is unsubstituted or substituted with a phenyl; or a C₆-C₁₀ aryl;
if m=2, then R₂ is a direct linkage or a —(CH₂)ₚ— radical with p being an integer from 1 to 3;
(iii) a UV screen of the dibenzoylmethane type.

2. Composition as defined by claim 1, wherein the amount of fragrancing substance is from 3% to 50% by weight, better still from 5% to 30% by weight, even better still from 10% to 20% by weight, relative to the total weight of the composition.

3. Composition as defined by claim 1, wherein the compound A of formula (I) is n-hexyl 2-(4-diethylamino-2-hydroxybenzoyl)benzoate of formula (A) below:

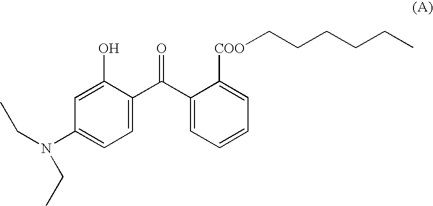

4. Composition as defined by claim 1, wherein the cinnamate compound B is ethylhexyl methoxycinnamate of formula (B) below:

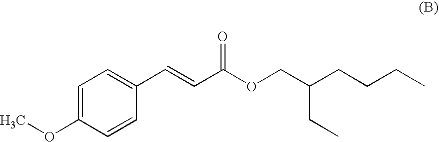

5. Composition as defined by claim 1, wherein the composition contains the n-hexyl 2-(4-diethylamino-2-hydroxybenzoyl)benzoate/ethylhexyl methoxycinnamate combination in a ratio by weight of 35/65% by weight.

6. Composition as defined by claim 1, wherein the piperidinol compound C of formula (II) is tris(tetramethylhydroxypiperidinol) citrate having the structure: 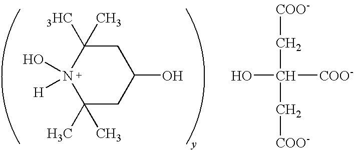
with y=3.

7. Composition as defined by claim 1, wherein the benzotriazole compound C of formula (III) is selected from among the following compounds:
(i) sodium benzotriazolyl butylphenol sulfonate of formula (I) 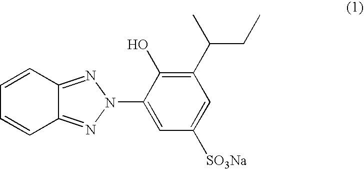
(ii) benzotriazolyl dodecyl p-cresol of formula (2): 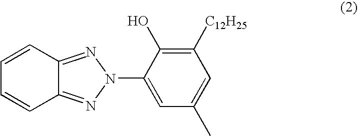
(iii) benzotriazolyl butylphenol sulfonate of formula (3): 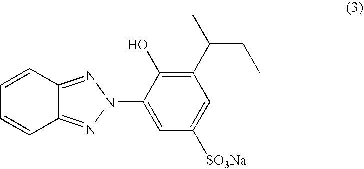
(iv) bumetrizole of formula (4):

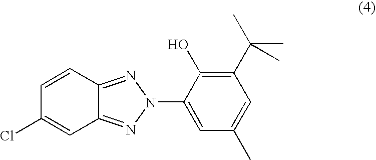

8. Composition as defined by claim 7, wherein the compound C of formula (III) is bumetrizole of formula (4):

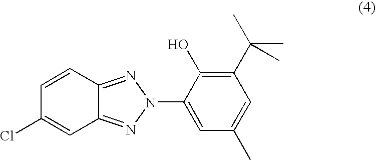

9. Composition as defined by claim 1, wherein the compound C is a dibenzoylmethane and that it also contains at least one salicylate screen D.

10. Composition as defined by claim 1, wherein the dibenzoylmethane compound C is 4-(tert-butyl)-4′-methoxydibenzoylmethane or butyl methoxydibenzoylmethane of formula (IV) below:

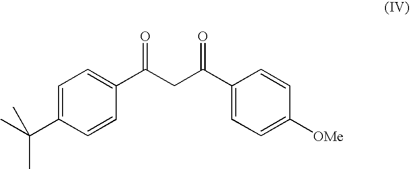

11. Composition as defined by claim 1, wherein the salicylate screen D is ethylhexyl salicylate of formula (V) below:

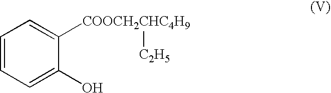

12. Composition as defined by claim 1, wherein the cosmetically acceptable medium contains at least one volatile alcohol and/or one volatile silicone oil and, optionally, water.

13. Composition as defined by claim 12, wherein the cosmetically acceptable medium contains water in an amount preferably ranging from 0.01% to 50%, and more preferably from 0.5% to 25% by weight, relative to the total weight of the composition.

14. Use of the combination of at least one screen A of the hydroxyaminobenzophenone type of formula (I), at least one screen B of the cinnamate type and at least one compound C as defined in the preceding claims, in a fragrancing composition comprising, in a cosmetically acceptable medium, at least 2% by weight of a fragrancing substance, relative to the total weight of the composition, as an agent for stabilizing the organoleptic properties of the composition with respect to outside attacks such as the light or temperature differences, in particular the color and/or the odor of said composition.

15. Cosmetic process for fragrancing human keratin materials, comprising the application of the composition as defined by claim 1, to the keratin materials.
